(19) United States
(12) Patent Application Publication
(10) Pub. No.: US 2018/0139920 A1
(43) Pub. Date: May 24, 2018

(54) WATERMELON VARIETY NUN 31503 WMW

(71) Applicant: Nunhems B.V., Nunhem (NL)

(72) Inventor: Yen Ming Chang, Lodi, CA (US)

(21) Appl. No.: 15/874,940

(22) Filed: Jan. 19, 2018

Related U.S. Application Data
(60) Provisional application No. 62/450,609, filed on Jan. 26, 2017.

(51) Int. Cl. A01H 5/08; C12N 15/82

(52) U.S. Cl. CPC A01H 5/08; C12N 15/8286; C12N 15/8274; C12N 15/8289

(57) ABSTRACT

The invention provides a new and distinct hybrid variety of Watermelon, NUN 31503 WMW, as well as seeds and plants and fruits thereof.

### CROSS-REFERENCE TO RELATED APPLICATIONS

The present application claims the benefit of U.S. Patent Application Ser. No. 62/450,609, filed 26 Jan. 2017, which is incorporated herein by reference in its entirety.

### FIELD OF THE INVENTION

The present invention relates to the field of plant breeding and, more specifically, to the development of NUN 31503 WMW (also designated as NUN 31503 or NUN 31503 triploid). The invention further relates to vegetative reproductions of NUN 31503 WMW, methods for tissue culture of NUN 31503 WMW and regenerating a plant from such a tissue culture and also to phenotypic variants of NUN 31503 WMW.

The goal of vegetable breeding is to combine various desirable traits in a single variety. Such desirable traits may include greater yield, resistance to diseases, insects or other pests, tolerance to heat and drought, better agronomic quality, higher nutritional value, enhanced growth rate and improved fruit properties. Breeding techniques take advantage of a plant's method of pollination. There are two general methods of pollination: a plant self-pollinates if pollen from one flower is transferred to the same or another flower of the same genotype. A plant cross-pollinates if pollen comes to it from a flower of a different genotype.

Plants that have been self-pollinated and selected for (uniform) type over many generations become homozygous at almost all gene loci and produce a uniform population of true breeding progeny of homozygous plants. The development of uniform varieties requires the development of homozygous inbred plants, the crossing of these inbred plants to make hybrids, and the evaluation of the hybrids resulting from the crosses. Pedigree breeding and recurrent selection are examples of breeding methods that have been used to develop inbred plants from breeding populations. Those breeding methods combine the genetic backgrounds from two or more plants or various other broad-based sources into breeding pools from which new lines are developed by selfing and selection of desired phenotypes. The new plants are evaluated to determine which have commercial potential. One crop species which has been subject to such breeding programs and is of particular value is the Watermelon.

One valuable crop which has been subject to breeding programs is Watermelon, a member of the Cucurbitacea family. The genus Citrullus originated in Africa. The plant is a large and sprawling annual, grown for its fruit. Watermelon fruit is often colored attractively, commonly red. Watermelon can contain black seeds, which are considered undesirable for certain uses.

Many different watermelon cultivars have been produced, and watermelon breeding efforts have been underway in many parts of the world. Breeding objectives include varying the color, texture and flavor of the fruit, absence of seeds, disease or pest resistance, optimizing flesh thickness, yield, suitability to various climatic circumstances, solid content (% dry matter), and sugar content.

Watermelon (Citrullus lanatus) can occur as a diploid, triploid or tetraploid. Seedless watermelon fruits are produced by using pollen from diploid male parent plants to fertilize flowers of tetraploid maternal parent plants. Pollination of the tetraploid flowers with diploid pollen leads to hybrid F1 seeds which are triploid (Kihara, 1951, Proceedings of American Society for Horticultural Science 58: 217-230; Eigsti 1971, Hort Science 6: 1-2). The triploid hybrid plants grown from these F1 seeds are self-infertile as they produce sterile pollen due to chromosome imbalance, and need to be pollinated by a diploid pollenizer to produce watermelon fruit. Triploid plants are, therefore, interplanted with pollenizer plants for fruit production. The “seedless” fruit produced after pollination on the triploid hybrid plant are not truly seedless, but often contain some undeveloped, small, pale seeds, which are edible. Plants are generally planted at a ratio of 1 pollenizer per every 2-4 triploid plants. Triploid plants and pollenizers are either planted in separate rows (e.g. 1 row of pollenizer and 2-4 rows of triploids), or interplanted within rows (e.g. planting 1 pollenizer plant in between 2 to 3 triploid plants in the same row), or interplanted in narrow rows between rows of triploids (see a.o. Table 2 of US2006/0168701). The fruit produced on the pollenizer plants preferably has a different rind pattern from the fruit on the triploid hybrids, so that these can be easily distinguished.

Although hybrid triploid (seedless) watermelons have been grown in the United States for many decades, there is still a need for improved varieties. Consumer demand is high, and the seedless fruit of triploid watermelons are very desired, both for the fresh and the processed market. Many different triploid watermelon varieties exist (available on the world wide web at cuke.hort.ncsu.edu/ which can be downloaded from cucurbit/wmelon/wmcultab.html), producing fruits of different sizes and shapes, as well as different fruit quality. Grading of fruits is usually done by fruit weight, to distinguish “mini” watermelons, with weights of less than 6 pounds (2.72 kg), “icebox” watermelons with weights of 8-12 pounds (3.62 kg-5.44 kg) or, according to others, of 6 to 15 pounds (2.72 kg to 6.8 kg) and “picnic” watermelons of above the icebox size, so either above 12 lb (above 5.44 kg) or above 15 pounds (above 6.8 kg). Furthermore, watermelon fruit flesh can have various colors, including various tints of red, pink, orange and yellow.

### SUMMARY OF THE INVENTION

In an aspect of the invention, a seed of Watermelon variety NUN 31503 WMW is provided, wherein a representative sample of said seed will be deposited under Accession Number NCIMB ______. The invention also provides for a plurality of seeds of NUN 31503 WMW. The Watermelon seed of NUN 31503 WMW may be provided as an essentially homogeneous population of Watermelon seed. Therefore, seed of the invention may be defined as forming at least about 97% of the total seed, including at least about 98%, 99% or more of the seed. The population of seed of NUN 31503 WMW may be particularly defined as being essentially free from other seed. The seed population may be grown into plants to provide an essentially homogeneous population of Watermelon plants according to the invention.

Also encompassed is a plant grown from a seed of Watermelon variety NUN 31503 WMW and a plant part thereof. In another aspect the invention provides for a variety of Watermelon called NUN 31503 WMW. The invention also provides for a progeny of NUN 31503 WMW. Especially, a plant or a progeny retaining all or all but one, two or three of the “morphological and physiological characteristics” of NUN 31503 WMW referred to herein, is encompassed herein as well as methods for producing that plant or progeny.

In one aspect, a plant or a progeny of the invention have all the physiological and morphological characteristics of variety NUN 31503 WMW when grown under the same environmental conditions. In another aspect such a plant or such progeny have all or all but one, two or three of the physiological and morphological characteristics of NUN 31503 WMW when measured under the same environmental conditions and evaluated at significance levels of 1%, 5% or 10% significance (which can also be expressed as a p-value) wherein a representative sample of seed of variety NUN 31503 WMW will be deposited under Accession Number NCIMB ______. In a second aspect, a plant or a progeny of the invention have all the physiological and morphological characteristics of variety NUN 31503 WMW when grown under the same environmental conditions. In another aspect such a plant or such progeny have all or all but one, two or three of the physiological and morphological characteristics as listed in Table 1 and/or 2 for variety NUN 31503 WMW when measured under the same environmental conditions and evaluated at significance levels of 1%, 5% or 10% significance.

In another aspect a plant of NUN 31503 WMW or said progeny plants has 5, 6, or more or all of the distinguishing characteristics (averages): 1) average mature fruit length; 2) average mature fruit weight; 3) average mature fruit diameter; 4) blossom end rind thickness; 5) median rind thickness; 6) Refractometer % of soluble solids of juice (centre of fruit); 7) Flower diameter across staminate; and 8) petiole length. NUN 31503 WMW is a triploid Watermelon and does not form seed. The market segment is “personal size”.

Also a plant part obtained from variety NUN 31503 WMW is provided, wherein said plant part is selected from the group consisting of: a fruit, a harvested fruit, a part of a fruit, a leaf, a part of a leaf, pollen, an ovule, a cell, a petiole, a shoot or a part thereof, a stem or a part thereof, a root or a part thereof, a root tip, a cutting, a seed of NUN 31503 WMW, a part of a seed of NUN 31503 WMW, hypocotyl, cotyledon, a scion, a stock, a rootstock, a pistil, an anther, and a flower or a part thereof. Fruits are particularly important plant parts. In a further embodiment, the plant part obtained from variety NUN 31503 WMW is a cell, optionally a cell in a cell or tissue culture. That cell may be grown into a plant of NUN 31503 WMW.

The invention also provides a cell culture of NUN 31503 WMW and a plant regenerated from NUN 31503 WMW, which plant has all the characteristics of NUN 31503 WMW when grown under the same environmental conditions, as well as methods for regenerating NUN 31503 WMW. Alternatively, a regenerated plant may have one characteristic that is different from NUN 31503 WMW.

Further, a vegetatively propagated plant of variety NUN 31503 WMW is provided having all or all but one, two or three of the morphological and physiological characteristics NUN 31503 WMW when grown under the same environmental conditions.

Further, a Watermelon fruit produced on a plant grown from a seed of NUN 31503 WMW is provided.

In still another aspect, a seed growing or grown on a plant of NUN 31503 WMW is provided (i.e. produced after pollination of the flower of NUN 31503 WMW).

### Definitions

All patent and non-patent documents cited herein are incorporated by reference in their entirety.

“Watermelon” refers herein to plants of the species Citrullus lanatus. The most commonly eaten part of a Watermelon is the fruit. The fruit comprises a stem and peduncle or pedicel, receptacle, ectocarp, rind, fruit flesh, exocarp, mesocarp, external phloem, internal phloem, xylem, vascular bundle, carpel, placenta and optionally seed. The stem and peduncle or pedicel, receptacle, ectocarp, rind, fruit flesh, exocarp, mesocarp, external phloem, internal phloem, xylem, vascular bundle, carpel, placenta and seedcoat of the seed are maternal tissues, that is they are genetically identical to the plant on which they grow.

“Cultivated Watermelon” refers to plants of Citrullus lanatus i.e. varieties, breeding lines or cultivars of the species C. lanatus, cultivated by humans and having good agronomic characteristics; preferably such plants are not “wild plants”, i.e. plants which generally have much poorer yields and poorer agronomic characteristics than cultivated plants and e.g. grow naturally in wild populations. “Wild plants” include for example ecotypes, PI (Plant Introduction) lines, landraces or wild accessions or wild relatives of Watermelon.

The terms “Watermelon plant designated NUN 31503 WMW”, “NUN 31503 WMW”, “NUN 31503”, “NUN 31503 F1”, “31503 WMW” or “Watermelon 31503” are used interchangeably herein and refer to a Watermelon plant of variety NUN 31503 WMW, representative seed of which will be deposited under Accession Number NCIMB ______.

A “seed of NUN 31503 WMW” refers to a Watermelon seed which can be grown into a plant of NUN 31503 WMW wherein a representative sample of viable seed of NUN 31503 WMW will be deposited under Accession Number NCIMB ______. A seed can be in any stage of maturity, for example a mature, viable seed, or an immature, non-viable seed. A seed comprises an embryo and maternal tissues.

An “embryo of NUN 31503 WMW” refers to an “F1 hybrid embryo” as present in a seed of NUN 31503 WMW, a representative sample of said seed of NUN 31503 WMW will be deposited under Accession Number NCIMB ______. The embryo is a triploid hybrid.

A “fruit of NUN 31503 WMW” refers to a fruit containing maternal tissues of NUN 31503 WMW as deposited under Accession Number NCIMB ______. The fruit of NUN 31503 WMW is seedless, as is usual for triploid watermelons. The skilled person is also familiar with methods for inducing parthenocarpy. Those methods comprise chemically or genetically inducing parthenocarpy, or by use of irradiated pollen (Moussa and Salem, 2010). Compounds suitable for chemically inducing parthenocarpy include auxins, gibberellins and cytokinins (see a.o. Hayata et al., 1995). A fruit can be in any stage of maturity, for example a mature fruit in the stage comprising viable seed, or an immature fruit comprising non-viable seed. In watermelon, growing triploid plants to produce seedless fruits is particularly preferred, see a.o. WO2010111031.

“Tissue culture” or “cell culture” refers to a composition comprising isolated cells of the same or a different type or a collection of such cells organized into parts of a plant. Tissue culture of various tissues of watermelon and regeneration of plants therefrom is well known and widely published (see, e.g., Compton et al., Plant Cell, Tissue and Organ Culture 77: 231-243, 2004). Similarly, the skilled person is well-aware how to prepare a “cell culture”.

“UPOV descriptors” are the plant variety descriptors described for watermelon in the “Guidelines for the Conduct of Tests for Distinctness, Uniformity and Stability, TG/142/5 (Geneva, 2013), as published by UPOV (International Union for the Protection of New Varieties and Plants, available on the world wide web at upov.int) and which can be downloaded from the world wide web at upov.int/ under edocs/tgdocs/en/tg142.pdf and is herein incorporated by reference in its entirety.

“USDA descriptors” are the plant variety descriptors for watermelon (Citrullus lanatus) as published by the US Department of Agriculture, Agricultural Marketing Service, Plant Variety Protection Office, Beltsville, Md. 20705 (available on the world wide web at ams.usda.gov) and which can be downloaded from the world wide web at /sites/default/files/media/18-Watermelon%20ST-470-18%202015.pdf.

“RHS” refers to the Royal Horticultural Society of England which publishes an official botanical color chart quantitatively identifying colors according to a defined numbering system. The chart may be purchased from Royal Horticulture Society Enterprise Ltd RHS Garden; Wisley, Woking; Surrey GU236QB, UK, e.g., the RHS colour chart: 2007 (The Royal Horticultural Society, charity No: 222879, PO Box 313 London SW1P2PE.

As used herein and except as otherwise indicated, the term “plant” includes the whole plant or any part thereof, preferably having the same genetic makeup as the plant from which it is obtained, such as a plant organ (e.g. harvested or non-harvested fruits), a plant cell, a plant protoplast, a plant cell tissue culture or a tissue culture from which a whole plant can be regenerated, a plant cell that is intact in a plant, a clone, a micropropagation, plant callus, a plant cell clump, a plant transplant, a vegetative propagation, a seedling, or parts of a plant (e.g. harvested tissues or organs), such as a fruit, a harvested fruit, a part of a fruit, a leaf, a part of a leaf, pollen, an ovule, an embryo, a petiole, a shoot or a part thereof, a stem or a part thereof, a root or a part thereof, a root tip, a cutting, a seed, a part of a seed hypocotyl, cotyledon, a scion, a graft, a stock, a rootstock, a pistil, an anther, and a flower or parts of any of these and the like. Also any developmental stage is included, such as seedlings, cuttings prior or after rooting, mature plants or leaves.

“Harvested plant material” refers herein to plant parts (e.g. fruits detached from the whole plant) which have been collected for further storage and/or further use.

“Reference Variety” or “check variety” refers herein to variety Pixie, a commercial variety from company Nunhems B.V., which has been planted in a trial together with NUN 31503 WMW. USDA descriptors of NUN 31503 WMW were compared to the USDA descriptors of Pixie.

“Rootstock” or “stock” refers to the plant selected for its roots, in particular for the resistance of the roots to diseases or stress (e.g. heat, cold, salinity etc.). Normally the quality of the fruit of the plant providing the rootstock is less important.

“Scion” refers to a part of the plant that is attached to the rootstock. This plant is selected for its stems, leaves, flowers, or fruits. The scion contains the desired genes to be duplicated in future production by the stock/scion plant and may produce the desired Watermelon fruit.

“Stock/scion” plant refers to a Watermelon plant comprising a rootstock from one plant grafted to a scion from another plant.

A plant having “all the physiological and morphological characteristics” of a referred-to-plant means a plant showing the physiological and morphological characteristics of the referred-to-plant when grown under the same environmental conditions, preferably in the same experiment; the referred-to-plant can be a plant from which it was derived, e.g. the progenitor plant, the parent, the recurrent parent, the plant used for tissue- or cell culture, etc. A physiological or morphological characteristic can be a numerical characteristic or a non-numerical characteristic. In one aspect, a plant has “all but one, two or three of the physiological and morphological characteristics” of a referred-to-plant, or “all the physiological and morphological characteristics” of Table 1 and/or 2 or “all or all but one, two or three of the physiological and morphological characteristics” of Table 1 and/or 2.

The physiological and/or morphological characteristics mentioned above are commonly evaluated at significance levels of 1%, 5% or 10% if they are numerical, or for having an identical degree (or type) if not numerical, if measured under the same environmental conditions. For example, a progeny plant or a Single Locus Converted plant or a mutated plant of NUN 31503 WMW may have one or more (or all) of the essential physiological and/or morphological characteristics of said variety listed in Table 1 and/or 2, as determined at the 5% significance level (i.e. p<0.05) when grown under the same environmental conditions.

“Distinguishing characteristics” or “distinguishing morphological and/or physiological characteristics” refers herein to the characteristics which distinguish (i.e. are different) between the new variety and other Watermelon varieties, such as the Reference Variety, when grown under the same environmental conditions. The distinguishing characteristics between NUN 31503 WMW and Reference Variety are described elsewhere herein and also can be seen in Table 1 and/or Table 2. When comparing NUN 31503 WMW with different varieties, the distinguishing characteristics will be different. In one aspect, the distinguishing characteristics may therefore include at least one, two, three or more (or all) of the characteristics listed in Table 1 and/or 2 and/or 3. All numerical distinguishing characteristics are statistically significantly different at p<0.05 between NUN 31503 WMW and the other variety, e.g. Reference Variety.

NUN 31503 WMW has the following distinguishing characteristics when compared to the Reference Variety Pixie: 1) average mature fruit length; 2) average mature fruit weight; 3) average mature fruit diameter; 4) blossom end rind thickness; 5) median rind thickness; 6) Refractometer % of soluble solids of juice (centre of fruit); 7) Flower diameter across staminate; and 8) petiole length. This can be seen in among others Table 1, where the USDA characteristics of NUN 31503 WMW are compared to the characteristics of Reference Variety, when grown under the same environmental conditions

Thus, a Watermelon plant “comprising the distinguishing characteristics of NUN 31503 WMW (such as a progeny plant) refers herein to a plant which does not differ significantly from said variety in the distinguishing characteristics above. Therefore in one aspect a plant (such as a progeny plant of NUN 31503 WMW) is provided which does not differ significantly from NUN 31503 WMW in the distinguishing characteristics above.

Similarity and differences between two different plant lines or varieties can be determined by comparing the number of morphological and/or physiological characteristics (e.g. the characteristics as listed in Table 1 and/or 2) that are the same (i.e. statistically not significantly different) or that are different (i.e. statistically significantly different) between the two plant lines or varieties when grown under the same environmental conditions. A numerical characteristic is considered to be “the same” when the value for a numeric characteristic is not significantly different at the 1% (p<0.01) or 5% (p<0.05) significance level, using one way Analysis of variance (ANOVA), a standard method known to the skilled person. Non-numerical or “degree” or “type” characteristic are considered “the same” when the values have the same “degree” or “type” when scored using USDA and/or UPOV descriptors, if the plants are grown under the same environmental conditions.

As used herein, the term “variety”, “cultivated Watermelon” or “cultivar” means a plant grouping within a single botanical taxon of the lowest known rank, which grouping, irrespective of whether the conditions for the grant of a breeder's right are fully met, can be defined by the expression of the characteristics resulting from a given genotype or combination of genotypes, distinguished from any other plant grouping by the expression of at least one of the said characteristics and considered as a unit with regard to its suitability for being propagated unchanged.

A “plant line” is for example a breeding line which can be used to develop one or more varieties. A breeding line is typically highly homozygous.

“Hybrid variety” or “F1 hybrid” refers to the seeds harvested from crossing two inbred (nearly homozygous) parental lines. For example, the female parent is pollinated with pollen of the male parent to produce hybrid (F1) seeds on the female parent. It also comprises triploid hybrids, where one parental line is tetraploid and the other parental line is diploid

“Regeneration” refers to the development of a plant from cell culture or tissue culture or vegetative propagation.

“Vegetative propagation”, “vegetative reproduction” or “clonal propagation” are used interchangeably herein and mean a method of taking a part of a plant and allowing that plant part to form at least roots, and also refer to the plant or plantlet obtained by that method. Optionally, the vegetative propagation is grown into a mature plant. The skilled person is aware of what plant parts are suitable for use in the method.

“Selfing” refers to self-pollination of a plant, i.e., the transfer of pollen from the anther to the stigma of the same plant.

“Crossing” refers to the mating of two parent plants. The term encompasses “cross-pollination” and “selfing”.

“Cross-pollination” refers to the fertilization by the union of two gametes from different plants.

“Yield” means the total weight of all Watermelon fruits harvested per hectare of a particular line or variety. It is understood that “yield” expressed as weight of all Watermelon fruits harvested per hectare can be obtained by multiplying the number of plants per hectare times the “yield per plant”. “Marketable yield” means the total weight of all marketable Watermelon fruits, especially fruit that is not cracked, damaged or diseased, harvested per hectare of a particular line or variety.

As used herein, the terms “resistance” and “tolerance” are used interchangeably to describe plants that show no symptoms or significantly reduced symptoms to a specified biotic pest, pathogen, abiotic influence or environmental condition compared to a susceptible plant. These terms are optionally also used to describe plants showing some symptoms but that are still able to produce marketable product with an acceptable yield.

“Harvest maturity” is referred to as the stage at which a Watermelon fruit is ripe or ready for harvest or the optimal time to harvest the fruit for the market, for processing or for consumption. In one embodiment, harvest maturity is the stage which allows proper completion of the normal ripening.

“Flavor” (or flavour) refers to the sensory impression of a food or other substance, especially a Watermelon fruit or fruit part (fruit flesh) and is determined mainly by the chemical senses of taste and smell. Flavor is influenced by texture properties and by volatile and/or non-volatile chemical components (organic acids, lipids, carbohydrates, salts etc.).

“Aroma” refers to smell (or odor) characteristics of Watermelon fruits or fruit parts (fruit flesh).

The term “traditional breeding techniques” encompasses herein crossing, selfing, selection, doubled haploid production, embryo rescue, protoplast fusion, marker assisted selection, mutation breeding etc. as known to the breeder (i.e. methods other than genetic modification/transformation/transgenic methods), by which, for example, a genetically heritable trait can be transferred from one Watermelon line or variety to another. It optionally includes epigenetic modifications.

“Backcrossing” is a traditional breeding technique used to introduce a trait into a plant line or variety. The plant containing the trait is called the donor plant and the plant into which the trait is transferred is called the recurrent parent. An initial cross is made between the donor parent and the recurrent parent to produce a progeny plant. Progeny plants which have the trait are then crossed to the recurrent parent. After several generations of backcrossing and/or selfing the recurrent parent comprises the trait of the donor. The plant generated in this way may be referred to as a “single trait converted plant”. The technique can also be used on a parental line of a hybrid.

“Progeny” as used herein refers to a plant obtained from a plant designated NUN 31503 WMW. A progeny may be obtained by regeneration of cell culture or tissue culture or parts of a plant of said variety or selfing of a plant of said variety or by producing seeds of a plant of said variety. A progeny may comprise a mutation or a transgene. A first generation progeny” or is the progeny directly derived from, obtained from, obtainable from or derivable from the parent plant by, e.g., regeneration. Progeny may have all the physiological and morphological characteristics of variety NUN 31503 WMW when grown under the same environmental conditions and/or progeny may have (be selected for having) one or more of the distinguishing characteristics of Watermelon of the invention.

The terms “gene converted” or “conversion plant” or “single locus converted plant” in this context refer to Watermelon plants which are developed by backcrossing wherein essentially all of the desired morphological and physiological characteristics of the parent variety or line are recovered, in addition to the one or more genes transferred into the parent via the backcrossing technique (optionally including reverse breeding or reverse synthesis of breeding lines) or via genetic engineering or through mutation breeding. Likewise a “Single Locus Converted (Conversion) Plant” refers to plants which are developed by plant breeding techniques comprising or consisting of backcrossing, wherein essentially all of the desired morphological and physiological characteristics of a Watermelon variety are recovered in addition to the characteristics of the single locus having been transferred into the variety via the backcrossing technique and/or by genetic transformation and/or by mutation. In case of a hybrid, the gene may be introduced in the male or female parental line.

“Marker” refers to a readily detectable phenotype, preferably inherited in codominant fashion (both alleles at a locus in a diploid heterozygote are readily detectable), with no environmental variance component, i.e., a heritability of 1.

“Average” refers herein to the arithmetic mean.

The term “mean” refers to the arithmetic mean of several measurements. The skilled person understands that the appearance of a plant depends to some extent on the growing conditions of said plant. Thus, the skilled person will know typical growing conditions for Watermelons described herein. The mean, if not indicated otherwise within this application, refers to the arithmetic mean of measurements on at least 10 different, randomly selected plants of a variety or line.

### DETAILED DESCRIPTION

The present invention relates to a plant of NUN 31503 WMW wherein a representative sample of seeds of said variety is deposited under the Budapest Treaty, with Accession number NCIMB ______.

The present invention also relates to a seed of Watermelon variety, referred to as NUN 31503 WMW, wherein a representative sample of said seed is deposited under the Budapest Treaty, with Accession number NCIMB ______.

In another aspect, the invention provides for a Watermelon plant part of variety NUN 31503 WMW, preferably a fruit, a representative sample of seed from said variety is deposited under the Budapest Treaty, with Accession number NCIMB ______.

A seed of variety NUN 31503 WMW is obtainable by crossing the male parent of said variety with the female parent of said variety and harvesting the seeds produced on the female parent. The resultant seed of NUN 31503 WMW can be grown to produce plants of said variety. In one embodiment a seed or a plurality of seeds of said variety are packaged into a container of any size or type (e.g., bag, carton, can, etc.). The seed may be disinfected, primed and/or treated with various compounds, such as seed coatings or crop protection compounds. The seed produces a plant of NUN 31503 WMW.

Also provided is a plant of Watermelon variety NUN 31503 WMW, or a fruit or other plant part thereof, produced from a seed, wherein a representative sample of said seed is deposited under the Budapest Treaty, with Accession Number NCIMB ______.

Also a plant part obtained from variety NUN 31503 WMW is provided, wherein said plant part is selected from the group consisting of: a fruit, a harvested fruit, a part of a fruit, a leaf, a part of a leaf, pollen, an ovule, a cell, a petiole, a shoot or a part thereof, a stem or a part thereof, a root or a part thereof, a root tip, a cutting, a seed, a part of a seed, hypocotyl, cotyledon, a scion, a stock, a rootstock, a pistil, an anther, and a flower or a part thereof. Fruits are particularly important plant parts. Fruits are parthenocarpic, or seedless, or contain immature and/or nonviable seeds. In a further embodiment, the plant part obtained from variety NUN 31503 WMW is a cell, optionally a cell in a cell or tissue culture. That cell may be grown into a plant of NUN 31503 WMW. A part of a variety of the invention, i.e. NUN 31503 WMW (or of progeny NUN 31503 WMW or of a plant having all physiological and/or morphological characteristics but one, two or three which are different from those of NUN 31503 WMW) further encompasses any cells, tissues, organs obtainable from the seedlings or plants in any stage of maturity.

The invention also provides for a food or feed product or a processed product comprising or consisting of a plant part described herein wherein the plant part can be identified as a part of the plant of the invention. Preferably, the plant part is a Watermelon fruit or part thereof and/or an extract from a fruit or another plant part described herein comprising at least one cell of NUN 31503 WMW. The food or feed product may be fresh or processed, e.g., dried, grinded, powdered, pickled, chopped, cooked, roasted, in a sauce, in a sandwich, pasted, puréed or concentrated, juiced, pickled, canned, steamed, boiled, fried, blanched and/or frozen, etc.

Such a plant part of NUN 31503 WMW can be stored and/or processed further. Encompassed are therefore also food or feed products comprising one or more of such parts, such as canned, chopped, cooked, roasted, in a sauce, in a sandwich, pasted, puréed or concentrated, juiced, frozen, dried, pickled, or powdered Watermelon fruit from NUN 31503 WMW or from progeny of said varieties, or from a derived variety, such as a plant having all but one, two or three physiological and/or morphological characteristics of NUN 31503 WMW.

In a preferred embodiment, the invention provides for a Watermelon fruit of triploid variety NUN 31503 WMW, or a part of a fruit of said varieties. The fruit can be in any stage of maturity, for example immature or mature. In another embodiment, the invention provides for a container comprising or consisting of a plurality of harvested Watermelon fruits or parts of fruits of said variety, or fruits of progeny thereof, or fruits of a derived variety.

In another embodiment the plant, plant part or seed of NUN 31503 WMW is inside a container, For example, containers such as cans, boxes, crates, bags, cartons, Modified Atmosphere Packagings, films (e.g. biodegradable films), etc. comprising a plant or a part of a plant (fresh and/or processed) of NUN 31503 WMW or a seed of NUN 31503 WMW are also provided herein. In a preferred embodiment, the container comprises a plurality of seeds of NUN 31503 WMW, or a plurality of plant parts of NUN 31503 WMW.

The present invention further relates to a Watermelon variety, referred to as NUN 31503 WMW which—when compared to its REFERENCE VARIETY Pixie—has the following distinguishing characteristics: 1) average mature fruit length; 2) average mature fruit weight; 3) average mature fruit diameter; 4) blossom end rind thickness; 5) median rind thickness; 6) Refractometer % of soluble solids of juice (center of fruit); 7) Flower diameter across staminate; and 8) petiole length, where the characteristics are determined at the 5% significance level for plants grown under the same environmental conditions. Also encompassed by the present invention are parts of that plant.

In one embodiment a plant of NUN 31503 WMW or a progeny plant thereof, comprises all of the following morphological and/or physiological characteristics (i.e. average values of distinguishing characteristics, as indicated on the USDA Objective description of variety—Watermelon (unless indicated otherwise)): 1) average mature fruit length; 2) average mature fruit weight; 3) average mature fruit diameter; 4) blossom end rind thickness; 5) median rind thickness; 6) Refractometer % of soluble solids of juice (centre of fruit); 7) Flower diameter across staminate; and 8) petiole length, where the characteristics are determined at the 5% significance level for plants grown under the same environmental conditions. An example of values for the distinguishing characteristics collected in a trial run according to UDSA requirements can be found in Table 1. A part of this plant is also provided.

In another embodiment NUN 31503 WMW has resistance to Colletotrichum lagenarium race 1 that is 9—present (scale 1—absent/9—present) measured according to UPOV standards.

The invention further provides a Watermelon plant which does not differ from the plant of NUN 31503 WMW as determined at the 1%, 2%, 3%, 4% or 5% significance level when grown under the same environmental conditions. Thus the plants are measured in the same trial. Preferably, the trial is conducted as recommended by the USDA or UPOV. The invention also comprises a part of said plant

The invention also provides a tissue or cell culture comprising cells of NUN 31503 WMW. Such a tissue culture can be for example be grown on plates or in liquid culture, or be frozen for long term storage. The cells of NUN 31503 WMW used to start the culture can be selected from any plant part suitable for vegetative reproduction, or in a preferred embodiment can be selected from embryos, meristems, cotyledons, hypocotyl, pollen, leaves, anthers, roots, root tips, pistil, petiole, flower, fruit, seed, stem and stalks of NUN 31503 WMW. In another preferred embodiment, the tissue culture does not contain somaclonal variation or has reduced somaclonal variation. The skilled person is familiar with methods to reduce somaclonal variation, including regular reinitiation.

In one embodiment the invention provides a Watermelon plant regenerated from the tissue or cell culture of NUN 31503 WMW, wherein the regenerated plant is not significantly different from NUN 31503 WMW in all, or all but one, two or three, of the physiological and morphological characteristics (determined at the 5% significance level when grown under the same environmental conditions). Optionally, the plant has one, two or three the physiological and morphological characteristics that are effected by a mutation, preferably an induced mutation, or by transformation. In another embodiment, the invention provides a Watermelon plant regenerated from the tissue or cell culture of NUN 31503 WMW, wherein the plant has all of the physiological and morphological characteristics of said variety determined at the 5% significance level when grown under the same environmental conditions. In these cases, similarity or difference of a characteristic is determined by measuring the characteristics of a representative number of plants grown under the same environmental conditions, determining whether type/degree characteristics are the same or different and determining whether numerical characteristics are significantly different (determined at the 5% significance level).

A Watermelon according to the invention, such as NUN 31503 WMW, or its progeny, or a plant having all physiological and/or morphological characteristics but one, two or three which are different from those of NUN 31503 WMW, can also be reproduced using vegetative reproduction methods. Therefore, the invention provides for a method of producing a plant, or a part thereof, of variety NUN 31503 WMW, comprising vegetative propagation of said variety. Vegetative propagation comprises regenerating a whole plant from a plant part of variety NUN 31503 WMW (or from a progeny of said variety or from or a plant having all physiological and/or morphological characteristics of said variety but one, two or three different characteristics), such as a cutting, a cell culture or a tissue culture.

The invention also concerns methods of vegetatively propagating a part of the plant of the invention NUN 31503 WMW. In certain embodiments, the method comprises the steps of: (a) collecting tissue or cells capable of being propagated from a plant of the invention; (b) cultivating said tissue or cells to obtain proliferated shoots; and (c) rooting said proliferated shoots, to obtain rooted plantlets. Steps (b) and (c) may also be reversed, i.e. first cultivating said tissue to obtain roots and then cultivating the tissue to obtain shoots, thereby obtaining rooted plantlets. The rooted plantlets may then be further grown, to obtain plants. In one embodiment, the method further comprises step (d) growing plants from said rooted plantlets. Therefore, the method also comprises regenerating a whole plant from said part of NUN 31503 WMW.

In a preferred embodiment, the part of the plant to be propagated is is a cutting, a cell culture or a tissue culture.

The invention also provides for a vegetatively propagated plant of variety NUN 31503 WMW (or from or a plant having all but one, two or three physiological and/or morphological characteristics of NUN 31503 WMW) wherein the plant has all of the morphological and physiological characteristics of NUN 31503 WMW when the characteristics are determined at the 5% significance level for plants grown under the same conditions. In another embodiment, the propagated plant has all but one, two or three of the morphological and physiological characteristics of NUN 31503 WMW when the characteristics are determined at the 5% significance level for plants grown under the same conditions. A part of said propagated plant or said propagated plant with one, two or three differences is also included.

In an embodiment, the invention provides a method for producing a Watermelon plant, comprising the steps of:
a. Growing a plant of NUN 31503 WMW until it sets fruit
b. Collecting the fruit of step a)
   Preferably, the fruit is collected at harvest maturity. In another embodiment, the fruit is collected when the seed is ripe. A plant of NUN 31503 WMW can be produced by seeding directly in the soil (e.g., field) or by germinating the seeds in controlled environment conditions (e.g., greenhouses) and optionally then transplanting the seedlings into the field. For example, the seed can be sown into prepared seed beds where they will remain for the entire production the crop

The morphological and/or physiological differences between two different individual plants of the invention (e.g. between NUN 31503 WMW and a progeny of NUN 31503 WMW) or between a plant of NUN 31503 WMW or progeny of said variety, or a plant having all, or all but 1, 2, or 3, of the physiological and morphological characteristics of NUN 31503 WMW (or all, or all but 1, 2, or 3 of the characteristics as listed in Table 1 and/or 2) and another known variety can easily be established by growing said variety next to each other or next to the other variety (in the same field, under the same environmental conditions), preferably in several locations which are suitable for said Watermelon cultivation, and measuring morphological and/or physiological characteristics of a number of plants (e.g., to calculate an average value and to determine the variation range/uniformity within the variety). For example, trials can be carried out in Acampo Calif., USA (N 38 degrees 07′261″/W 121 degrees 18′807″, USA, whereby various characteristics, for example maturity, days from seeding to harvest, plant habit, plant attitude, leaf shape, leaf color, blistering, numbers of flowers per leaf axil, number of calyx lobes, number of petals, fruit group, immature fruit color, mature fruit color, pungency, flavor, fruit glossiness, fruit size, fruit shape, average number of fruits per plant, seed size, seed weight, anthocyanin level, disease resistance, insect resistance, can be measured and directly compared for species of Watermelon. Thus, the invention comprises Watermelon plant having one, two or three physiological and/or morphological characteristics which are different from those of the plant of NUN 31503 WMW and which otherwise has all the physiological and morphological characteristics of the plant of NUN 31503 WMW, when determined at the 5% significance level for plants grown under the same environmental conditions. In a preferred embodiment, the different characteristic is effected by a mutation, preferably an induced mutation, or by transformation.

The morphological and physiological characteristics (and the distinguishing characteristics) of NUN 31503 WMW are provided in the Examples, in Table 1 and/or 2. Encompassed herein is also a plant obtainable from NUN 31503 WMW (e.g. by selfings and/or crossing and/or backcrossing with said variety and/or progeny of said variety) comprising all or all but one, two or three of the physiological and morphological characteristics of NUN 31503 WMW listed in Table 1 and/or 2 as determined at the 5% significance level for numerical characteristics or identical for non-numerical characteristics when grown under the same environmental conditions and/or comprising one or more (or all; or all except one, two or three) characteristics when grown under the same environmental conditions.

Also at-harvest and/or post-harvest characteristics of fruits can be compared, such as cold storage holding quality, post-harvest flesh firmness, and Brix can be measured using known methods. (Fruit) Flesh firmness can for example be measured using a penetrometer, e.g. by inserting a probe into the fruit flesh and determining the insertion force, or by other methods. Fruit flesh firmness can for example be measured using a “FT 327 Penetrometer”, available from QA Supplies LLC, 1185 Pineridge Road, Norfolk, Va. 23502.

The morphological and/or physiological characteristics may vary somewhat with variation in the environment (such as temperature, light intensity, day length, humidity, soil, fertilizer use), which is why a comparison under the same environmental conditions is preferred. Colors can best be measured against The Munsell Book of Color (Munsell Color Macbeth Division of Kollmorgan Instruments Corporation) or using the Royal Horticultural Society Chart (World Wide Web at rhs.org.uk/Plants/RHS-Publications/RHS-colour-charts).

In a preferred embodiment, the invention provides for a Watermelon fruit of variety NUN 31503 WMW, or a part of a fruit of said varieties. In another embodiment, the invention provides for a container comprising or consisting of a plurality of harvested Watermelon fruits or parts of fruits of said variety, or fruits of progeny thereof, or fruits of a derived variety.

The invention provides for methods of producing plants which retain all the morphological and physiological characteristics of a plant of the invention i.e. NUN 31503 WMW. The invention provides also for methods of producing a plant comprising all but 1, 2, or 3 or more of the morphological and physiological characteristics of NUN 31503 WMW (e.g. as listed in Table 1 and/or 2), but which are still genetically closely related to said variety. The relatedness can, for example be determined by fingerprinting techniques (e.g., making use of isozyme markers and/or molecular markers such as Single-nucleotide polymorphism (SNP) markers, amplified fragment length polymorphism (AFLP) markers, microsatellites, minisatellites, Random Amplified Polymorphic DNA (RAPD) markers, restriction fragment length polymorphism (RFLP) markers and others). A plant is “closely related” to NUN 31503 WMW if its DNA fingerprint is at least 80%, 90%, 95% or 98% identical to the fingerprint of NUN 31503 WMW. In a preferred embodiment AFLP markers are used for DNA fingerprinting (Vos et al. 1995, Nucleic Acid Research 23: 4407-4414). A closely related plant may have a Jaccard's Similarity index of at least about 0.8, preferably at least about 0.9, 0.95, 0.98 or more (Gama et al., 2013. Horticultura Brasileira 31: 522-527).

The invention also provides a plant and a variety obtained or selected by applying these methods on NUN 31503 WMW. Such a plant may be produced by crossing and/or selfing, or alternatively, a plant may simply be identified and selected amongst plants of said variety, or progeny of said variety, e.g. by identifying a variant within NUN 31503 WMW or within progeny of said variety which variant differs from NUN 31503 WMW in one, two or three of the morphological and/or physiological characteristics (e.g. in one, two or three distinguishing characteristics), e.g. those listed in Table 1 and/or 2 or others. In one embodiment the invention provides a Watermelon plant having a Jaccard's Similarity index with NUN 31503 WMW of at least 0.8, e.g. at least 0.85, 0.9, 0.95, 0.98 or even at least 0.99.

WO2013182646 which is incorporated by reference, relates to a non-destructive method for analyzing maternal DNA of a seed. In this method the DNA is dislodged from the seed coat surface and can be used to collect information on the genome of the maternal parent of the seed. This method for analyzing maternal DNA of a seed, comprises the steps of contacting a seed with a fluid to dislodge DNA from the seed coat surface, and analyzing the DNA thus dislodged from the seed coat surface using methods known in the art. The seed coat of the seed is a maternal tissue genetically identical to the female parent. Thus the skilled person can determine the genetics of the female parent of NUN 31503 WMW.

Alternatively, a single trait converted plant or single locus converted plant or a plant having one, two or three characteristics that are different to those of of NUN 31503 WMW may be produced by the following steps
a. obtaining a cell or tissue culture of cells of NUN 31503 WMW;
b. genetically transforming or mutating said cells;
c. growing the cells into a plant; and
d. optionally selecting a plant that contains the desired single locus conversion or a desired trait
   The skilled person is familiar with various techniques for genetically transforming a single locus in a plant cell, or mutating said cells.

Any pest or disease resistance genes may be introduced into a plant according to the invention, i.e. NUN 31503 WMW, progeny of said variety or into a plant comprising all but 1, 2, or 3 or more of the morphological and physiological characteristics of NUN 31503 WMW (e.g. as listed in Table 1). Resistance to one or more of the following diseases or pests is preferably introduced into plants of the invention: Anthracnose, Downy Mildew, Fusarium Wilt, Gummy Stem Blight, Squash Mosaic, Watermelon Mosaic, Powdery Mildew, Cucumber Mosaic, Sunburn and Root Knot. Other resistance genes, against pathogenic viruses, fungi, bacteria, nematodes, insects or other pests may also be introduced.

For breeding methods in general see Principles of Plant Genetics and Breeding, 2007, George Acquaah, Blackwell Publishing, ISBN-13: 978-1-4051-3646-4.

In one embodiment, a plant according to the invention, i.e. NUN 31503 WMW may also be mutated (by e.g. irradiation, chemical mutagenesis, heat treatment, etc.) and mutated seeds or plants may be selected in order to change one or more characteristics of said variety. Methods such as TILLING may be applied to Watermelon populations in order to identify mutants. Similarly, NUN 31503 WMW may be transformed and regenerated, whereby one or more chimeric genes are introduced into the variety or into a plant comprising all but 1, 2, 3, or more of the morphological and physiological characteristics (e.g. as listed in Table 1 and/or 2). Transformation can be carried out using standard methods, such as Agrobacterium tumefaciens mediated transformation or biolistics, followed by selection of the transformed cells and regeneration into plants. A desired trait (e.g. genes conferring pest or disease resistance, herbicide, fungicide or insecticide tolerance, etc.) can be introduced into NUN 31503 WMW, or progeny of said variety, by transforming said variety or progeny of said variety with a transgene that confers the desired trait, wherein the transformed plant retains all or all but one, two or three of the phenotypic and/or morphological and/or physiological characteristics of NUN 31503 WMW or the progeny of said variety and contains the desired trait.

The invention also provides a plant or a cell of a plant comprising a desired trait produced by mutating a plant of variety NUN 31503 WMW or a cell thereof and selecting a plant the desired trait, wherein the mutated plant retains all or all but one of the phenotypic and morphological characteristics of said variety, optionally as described for each variety in in Table 1 and/or 2, and contains the desired trait and wherein a representative sample of seed of variety NUN 31503 WMW is deposited under Accession Number NCIMB ______. In a further embodiment, the desired trait is selected from the group consisting of yield, compact Watermelon, fruit quality, male sterility, herbicide tolerance, insect resistance, pest resistance, disease resistance, Powdery mildew resistance without necrosis, environmental stress tolerance, modified carbohydrate metabolism, modified protein metabolism and ripening.

A suitable method for inducing mutation in NUN 31503 WMW comprises the steps of:
a. Exposing a seed, a plant or a plant part or a cell of NUN 31503 WMW to a mutagenic chemical or to radiation, wherein a representative sample of seed of NUN 31503 WMW is deposited under Accession Number NCIMB ______.
b. Selecting a seed, a plant or a plant part or a cell of NUN 31503 WMW having a mutation
c. Optionally growing and/or multiplying the seed, plant or plant part or cell of NUN 31503 WMW having the mutation.

The invention also provides a plant having one, two or three physiological and/or morphological characteristics which are different from those of NUN 31503 WMW and which otherwise has all the physiological and morphological characteristics of said variety, wherein a representative sample of seed of variety NUN 31503 WMW is deposited under Accession Number NCIMB ______. In particular variants which differ from NUN 31503 WMW in none, one, two or three of the characteristics mentioned in Table 1 and/or 2 are encompassed.

A part of a variety of the invention, i.e. NUN 31503 WMW (or of progeny of said varieties or of a plant having all physiological and/or morphological characteristics but one, two or three which are different from those of said variety) encompasses any cells, tissues, organs obtainable from the seedlings or plants, such as but not limited to: a Watermelon fruit or a part thereof, a cutting, hypocotyl, cotyledon, pollen and the like. Such parts can be stored and/or processed further. Encompassed are therefore also food or feed products comprising a part of NUN 31503 WMW or a part of progeny of said varieties, or a part of a plant having all but one, two or three physiological and/or morphological characteristics of NUN 31503 WMW, comprising one or more of such parts, optionally processed (such as canned, chopped, cooked, roasted, in a sauce, in a sandwich, pasted, puréed or concentrated, juiced, frozen, dried, pickled, or powdered).

In one aspect a haploid plant and/or a doubled haploid plant of NUN 31503 WMW, or of a plant having all but one, two or three physiological and/or morphological characteristics of NUN 31503 WMW, or progeny of any of these, is encompassed herein. Haploid and doubled haploid (DH) plants can, for example, be produced by cell or tissue culture and chromosome doubling agents and regeneration into a whole plant. For DH production chromosome doubling may be induced using known methods, such as colchicine treatment or the like.

In yet another aspect haploid plants and/or doubled haploid plants derived from NUN 31503 WMW that, when combined, make a set of parents of NUN 31503 WMW are encompassed herein. Thus the haploid plant and/or the doubled haploid plant of NUN 31503 WMW can be used in a method for generating parental lines of NUN 31503 WMW.

Thus in one aspect, the invention relates to a method of producing a combination of parental lines of a plant of the invention (NUN 31503 WMW) comprising the step of making doubled haploid cells from haploid cells from said plant or a seed of that plant; and optionally crossing these parental lines to produce and collect seeds. In another aspect, the invention relates to a combination of parental lines produced by this method. In still another aspect said combination of parental lines can be used to produce a seed or plant of NUN 31503 WMW when these parental lines are crossed. In still another aspect, the invention relates to a combination of parental lines from which a seed or plant having all physiological and/or morphological characteristics of NUN 31503 WMW (when the characteristics are determined at the 5% significance level for plants grown under the same conditions).

In another aspect, the invention comprises a method for making doubled haploid cells from haploid cells of NUN 31503 WMW according to various methods known to the skilled person. A suitable method is colchicine treatment.

In another alternative aspect, the invention provides a method of introducing a single locus conversion or single trait conversion or a desired trait into NUN 31503 WMW comprising:
a. obtain a combination of a parental lines of NUN 31503 WMW, optionally through reverse synthesis of breeding lines,
b. introduce a single locus conversion in at least one of the parents of step a;
c. crossing the converted parent with the other parent of step a to obtain seed of NUN 31503 WMW
   A combination of a male and a female parental line of NUN 31503 WMW can be generated by methods described herein, for example through reverse synthesis of breeding lines.

In an embodiment of the invention, Step b) of the above method—introduce a single locus conversion in at least one of the parents of step a—may be done through the following method:
i. obtaining a cell or tissue culture of cells of the parental line of NUN 31503 WMW;
ii. genetically transforming or mutating said cells;
iii. growing the cells into a plant; and
iv. optionally selecting plants that contain the single locus conversion, the single trait conversion or the desired trait.

In another embodiment of the invention, Step b) of the above method—introduce a single locus conversion in at least one of the parents of step a—may also be done through the following method:
i. crossing the parental line of NUN 31503 WMW with a second Watermelon plant comprising the single locus conversion, the single trait conversion or the desired trait;
ii. selecting F1 progeny plants that contain the single locus conversion, the single trait conversion or the desired trait;
iii. crossing said selected progeny plants of step ii with the parental line of step i, to produce a backcross progeny plant;
iv. selecting backcross progeny plants comprising the single locus conversion, the single trait conversion or the desired trait and otherwise all or all but one, two or three of the morphological and physiological characteristics the parental line of step i to produce selected backcross progeny plants; and
v. optionally repeating steps iii and iv one or more times in succession to produce selected second, third or fourth or higher backcross progeny plants comprising the single locus conversion, the single trait conversion or the desired trait and otherwise all or all but one, two or three of the morphological and physiological characteristics the parental line of step i to produce selected backcross progeny plants, when grown in the same environmental conditions.
   The invention further relates to plants obtained by this method.

The above method is provided, wherein the single locus conversion concerns a trait, wherein the trait is yield or pest resistance or disease resistance. In one embodiment the trait is disease resistance and the resistance is conferred to Anthracnose, Downy Mildew, Fusarium Wilt, Gummy Stem Blight, Squash Mosaic, Watermelon Mosaic, Powdery Mildew, Cucumber Mosaic, Sunburn and Root Knot. Other resistance genes, against pathogenic viruses, fungi, bacteria, nematodes, insects or other pests may also be introduced.

Thus, the invention also provides a combination of parental lines which, when crossed, produce a seed or plant having all physiological and/or morphological characteristics of NUN 31503 WMW but one, two or three which are different (when grown under the same environmental conditions), as well as a seed or plant having all physiological and/or morphological characteristics of NUN 31503 WMW but one, two or three which are different (when the characteristics are determined at the 5% significance level for plants grown under the same conditions).

Also provided is a plant part obtainable from variety NUN 31503 WMW or from progeny of said variety or from a plant having all but one, two or three physiological and/or morphological characteristics which are different from those of NUN 31503 WMW, or from a vegetatively propagated plant of NUN 31503 WMW (or from its progeny or from a plant having all or all but one, two or three physiological and/or morphological characteristics which are different from those of NUN 31503 WMW), being selected from the group consisting of a fruit, a harvested fruit, a part of a fruit, a leaf, a part of a leaf, pollen, an ovule, a cell, a petiole, a shoot or a part thereof, a stem or a part thereof, a root or a part thereof, a root tip, a cutting, a seed, a part of a seed, or hypocotyl, cotyledon, a scion, a stock, a rootstock, a pistil, an anther, and a flower or a part thereof.

In still yet another aspect, the invention provides a method of determining the genotype of a plant of the invention comprising the step of detecting in the genome (e.g., a sample of nucleic acids) of the plant at least a first polymorphism or an allele. The skilled person is familiar with many suitable methods of genotyping, detecting a polymorphism or detecting an allele including restriction fragment length polymorphism identification (RFLP) of genomic DNA, random amplified polymorphic detection (RAPD) of genomic DNA, amplified fragment length polymorphism detection (AFLP), polymerase chain reaction (PCR), DNA sequencing, allele specific oligonucleotide (ASO) probes, and hybridization to DNA microarrays or beads. Alternatively, the entire genome could be sequenced. The method may, in certain embodiments, comprise detecting a plurality of polymorphisms in the genome of the plant, for example by obtaining a sample of nucleic acid from a plant and detecting in said nucleic acids a plurality of polymorphisms. The method may further comprise storing the results of the step of detecting the plurality of polymorphisms on a computer readable medium

The invention also provides for a food or feed product comprising or consisting of a plant part described herein wherein the plant part can be identified as a part of the plant of the invention. Preferably, the plant part is a Watermelon fruit or part thereof and/or an extract from a fruit or another plant part described herein. The food or feed product may be fresh or processed, e.g., dried, grinded, powdered, pickled, chopped, cooked, roasted, in a sauce, in a sandwich, pasted, puréed or concentrated, juiced, pickled, canned, steamed, boiled, fried, blanched and/or frozen, etc.

For example, containers such as cans, boxes, crates, bags, cartons, Modified Atmosphere Packagings, films (e.g. biodegradable films), etc. comprising a plant or a parts of a plant (fresh and/or processed) described herein or a seed of NUN 31503 WMW are also provided herein.

Marketable Watermelon fruits are generally sorted by size and quality after harvest. Alternatively the Watermelon fruits can be sorted by expected shelf life, pH or Brix.

Watermelons may also be grown for use as rootstocks (stocks) or scions (cions). Typically, different types of Watermelons are grafted to enhance disease resistance, which is usually conferred by the rootstock, while retaining the horticultural qualities usually conferred by the scion. It is not uncommon for grafting to occur between cultivated Watermelon varieties and related Watermelon species. Methods of grafting and vegetative propagation are well-known in the art.

So in one aspect the invention relates to a plant comprising a rootstock or scion of NUN 31503 WMW.

All documents (e.g., patent publications) are herein incorporated by reference in their entirety.

### CITED REFERENCES

Acquaah, Principles of Plant Genetics and Breeding, 2007, Blackwell Publishing, ISBN-13: 978-1-4051-3646-4
Compton et al., Plant Cell, Tissue and Organ Culture 77: 231-243, 2004
Eigsti 1971, Hort Science 6: 1-2
Gama et al., 2013. Horticultura Brasileira 31: 522-527
Hayata et al., 1995
Kihara, 1951, Proceedings of American Society for Horticultural Science 58: 217-230
Moussa and Salem, 2010
Martin et al. 2008, Australian Journal of Crop Science 1(2): 43-46
Vos et al. 1995, Nucleic Acid Research 23: 4407-4414
Wijnkers et al., Nature Protocols Volume: 9, Pages: 761-772 (2014) DOI: doi:10.1038/nprot.2014.049
WO2010111031
WO2013182646
WO2014076249
US 2006/0168701
TG/142/5 (Geneva, 2013), world wide web at upov.int/under edocs/tgdocs/en/tg142.pdf
world wide web at /sites/default/files/media/18-Watermelon%20ST-470-18%202015.pdf.
Horticultural Society Chart (World Wide Web at rhs.org.uk/Plants/RHS-Publications/RHS-colour-charts).

### EXAMPLES

### Development of NUN 31503 WMW

The triploid NUN 31503 WMW was developed from a male and female proprietary inbred line of Nunhems. The female and male parents were crossed to produce hybrid (F1) seeds of NUN 31503 WMW The seeds of NUN 31503 WMW can be grown to produce hybrid plants and parts thereof (e.g. Watermelon fruit). The hybrid NUN 31503 WMW can be propagated by seeds or vegetative.

The hybrid variety is uniform and genetically stable. This has been established through evaluation of horticultural characteristics. Several hybrid seed production events resulted in no observable deviation in genetic stability. Coupled with the confirmation of genetic stability of the female and male parents the Applicant concluded that NUN 31503 WMW is uniform and stable.

### Deposit Information

A total of 2500 seeds of the hybrid variety NUN 31503 WMW will be deposited according to the Budapest Treaty by Nunhems B.V. on ______, at the NCIMB Ltd., Ferguson Building, Craibstone Estate, Bucksburn, Aberdeen AB21 9YA, United Kingdom (NCIMB). The deposit will be assigned NCIMB number ______. A deposit of NUN 31503 WMW and of the male and female parent line is currently maintained at Nunhems B.V.

Access to the deposits will be available during the pendency of this application to persons determined by the Director of the U.S. Patent Office to be entitled thereto upon request. Subject to 37 C.F.R. § 1.808(b), all restrictions imposed by the depositor on the availability to the public of the deposited material will be irrevocably removed upon the granting of the patent. The deposit will be maintained for a period of 30 years, or 5 years after the most recent request, or for the enforceable life of the patent whichever is longer, and will be replaced if it ever becomes nonviable during that period. Applicant does not waive any rights granted under this patent on this application or under the Plant Variety Protection Act (7 USC 2321 et seq.).

The most similar variety to NUN 31503 WMW is referred to as Reference Variety, a variety from Nunhems B.V. with the commercial name Pixie. In Table 1 a comparison between NUN 31503 WMW and the Reference Variety is shown based on a trial in the USA. Trial location Esparto, Calif., transplanting date: 7 Jun. 2017.

Two replications of 50 plants of each variety, from which 15 plants or plant parts were randomly selected, were used to measure characteristics. For numerical characteristics averages were calculated. For non-numerical characteristics the type/degree was determined. In Table 1 the USDA descriptors of NUN 31503 WMW (this application) and the Reference Variety (commercial variety) are listed.

In accordance with one aspect of the present invention, there is provided a plant having the physiological and morphological characteristics of NUN 31503 WMW as presented in Table 1.

TABLE 1
Objective description of varieties NUN 31503 WMW and Reference Variety

| | Application variety | Reference variety |
|---|---|---|
| USDA descriptor | NUN 31503 WMW | Pixie |
| 1. General Fruit Type | | |
| Fruit type: 1 = Oblong, 2 = Round Large, 3 = Round | 3 | 3 |
| small (icebox), 4 = Other | | |
| 2. Area of best adaptation | | |
| Region: 1 = Southern US, 2 = Northeast/Central US, 3 = | 4 | 4 |
| Southwest US, 4 = Most US Areas, 5 = Other | | |
| 3. Maturity | | |
| No. of days from emergence to anthesis | n.r. | n.r. |
| No. of days from pollination to maturity | n.r. | n.r. |
| Days Relative Maturity (as reported in seed catalogs) | 78-80 | 78-80 |
| Maturity category: 1 = early, 2 = medium, 3 = late | 1 | 1 |
| 4. Ploidy | | |
| 1 = diploid, 2 = tetraploid, 3 = triploid | 3 | 3 |
| 5. Plant | | |
| Cotyledon shape: 1 = flat, 2 = folded | n.r. | n.r. |
| Plant sex form: 1 = monoecious, 2 = andromonoecious | 1 | 1 |
| No. of main stems at crown | 8.3 | 8.5 |
| No. of flowers per plant at first fruit set: | | |
| Staminate: | n.r. | n.r. |
| Pistilate: | n.r. | n.r. |
| Perfect: | n.r. | n.r. |
| 6. STEM | | |
| Stem shape (cross section:) 1 = round, 2 = angular | 2 | 2 |
| Diameter (mm) at second node | 10.2 | 9.6 |
| Stem surface: 1 = glabrous, 2 = scabrous, 3 = pubescent, | 3 | 3 |
| 4 = bristled | | |
| Vine length (cm) (at last harvest) | n.r. | n.r. |
| No. of Internodes (at last harvest) | n.r. | n.r. |
| Ratio Vine length (cm): No of internodes (at last harvest) | n.r. | n.r. |
| 7. LEAF | | |
| Leaf shape: 1 = ovate, 2 = obovate, 3 = round | 1 | 1 |
| Leaf lobes: 1 = none, 2 = lobed | 2 | 2 |
| Leaf length (cm) | 13.97 | 13.87 |
| Leaf width (cm) | 16.2 | 15.3 |
| Leaf size: 1 = longer than wide, 2 = length-width equal, | 3 | 3 |
| 3 = wider than long | | |
| Dorsal surface pubescence: 1 = smooth, 2 = pubescent | 2 | 2 |
| Ventral surface pubescence: 1 = smooth, 2 = pubescent | 2 | 2 |
| Leaf color: 1 = light green, 2 = gray green, 3 = medium | n.r. | n.r. |
| green, 4 = dark green (Color chart value (RHS)) | | |
| 8. FLOWER | | |
| Diameter across Staminate (cm) | 33.9 | 29.9 |
| Diameter across Pistillate (cm) | n.r. | n.r. |
| Diameter across Perfect (cm) | n.a. | n.a. |
| Flower color: 1 = lemon yellow, 2 = yellow, 3 = orange, | 2 (Yellow 6b) | 2 (Yellow 9b) |
| 4 = Other (Color chart value (RHS)) | | |
| 9. MATURE FRUIT | | |
| Fruit shape: 1 = round, 2 = oval, 3 = cylindrical | 1 | 1 |
| Long (cm) | 20.7 | 18.4 |
| Diameter at midsection (cm) | 18.8 | 17.4 |
| Average weight (kg) | 4.4 | 3.3 |
| Maximum fruit weight (kg) | 6.8 | 4.3 |
| Index = length ÷ diameter × 10 | 11 | 10.6 |
| Fruit surface: 1 = smooth, 2 = slightly grooved, 3 = deeply | 1 | 1 |
| grooved | | |
| Skin color pattern: 1 = solid (one color), 2 = stripe, 3 = | 2 | 2 |
| mottle/net | | |
| Primary color: 1 = Yellow Green (Desert King), 2 = Light | 4 (Green 139D) | 4 (Green 139A) |
| Green (Charleston Gray), 3 = Medium Green (Sugar | | |
| baby), 4 = dark green (Florida Giant), 5 = Other (Color | | |
| chart value (RHS)) | | |
| Secondary color: 1 = Yellow Green, 2 = Light Green, 3 = | 2 (Green 139A) | 3 (Green 138A) |
| Medium green, 4 = dark green, 5 = Other (Color chart | | |
| value (RHS)) | | |
| 10. RIND | | |
| Rind texture: 1 = tender, 2 = brittle, 3 = tough | 3 | 3 |
| Thickness blossom end (mm) | 10.9 | 6.6 |
| Thickness sides (mm) | 10.6 | 8.8 |
| 11. FLESH | | |
| Flesh texture: 1 = crisp, 2 = soft | 1 | 1 |
| Flesh coarseness: 1 = course fibrous, 2 = fine—little fiber | 2 | 2 |
| Flesh color: 1 = white, 2 = yellow, 3 = orange, 4 = pink, | 5 (Orange 35A) | 5 (Red 44A) |
| 5 = red, 6 = dark red (Color chart value (RHS)) | | |
| Refractometer: % Soluble solids of juice (Center of fruit) | 13.3 | 13.8 |
| % Hollow heart | n.r. | n.r. |
| % Placental separation | n.r. | n.r. |
| % Transverse crack | n.r. | n.r. |
| 12. Seed | N.A.—seedless | N.A.—seedless |

TABLE 2

| | Application Variety | Reference Variety |
|---|---|---|
| Non-USDA descriptor | NUN 31503 WMW | Pixie |
| Leaf petiole length in cm | 10.57 | 12.57 |
| Rind stem end thickness in mm | 10.5 | 11.7 |
| Flesh firmness kg/cm2 | 2.35 | 2.45 |

Table 1 and 2 contain typical values. Values may vary due to environment. Other values that are substantially equivalent are also within the scope of the invention. N.A.=not applicable; n.r.=not recorded.

What is claimed is:

1. A plant, plant part or seed of triploid Watermelon variety NUN 31503 WMW, wherein a representative sample of seed of said variety is deposited under Accession Number NCIMB ______.

2. The plant part of claim 1, further defined as a leaf, a seed, a fruit, a scion, a root, a rootstock, cutting, flower or a part of any of these or a cell.

3. A seed that produces the plant of claim 1.

4. A Watermelon plant which does not significantly differ from the plant of claim 1 in all of the distinguishing characteristics selected from the group consisting of: 1) average mature fruit length; 2) average mature fruit weight; 3) average mature fruit diameter; 4) blossom end rind thickness; 5) median rind thickness; 6) Refractometer % of soluble solids of juice (centre of fruit); 7) Flower diameter across staminate; and 8) petiole length, or a part of said plant.

5. A Watermelon plant which does not differ from the plant of claim 1 as determined at the 5% significance level when grown under the same environmental conditions, or a part of said plant.

6. A tissue or cell culture comprising cells of the plant of claim 1.

7. The tissue or cell culture according to claim 6, comprising cells or protoplasts derived from a plant part selected from the group of plant parts suitable for vegetative reproduction, preferably from the group consisting of embryos, meristems, cotyledons, hypocotyl, leaves, anthers, roots, root tips, pistil, petiole, flower, fruit, seed, stem and stalks.

8. A Watermelon plant regenerated from the tissue or cell culture of claim 6 or 7, wherein the plant has all of the physiological and morphological characteristics of the plant of NUN 31503 WMW, or one characteristic that is different from NUN 31503 WMW, when the characteristics are determined at the 5% significance level for plants grown under the same environmental conditions, and wherein a representative sample of seed of NUN 31503 WMW is deposited under Accession Number NCIMB ______.

9. The plant of claim 8, where the different characteristic is effected by a mutation or by transformation.

10. A method of producing the plant of claim 1, comprising vegetative propagation of at least a part of NUN 31503 WMW, wherein a representative sample of seed of said variety is deposited under Accession Number NCIMB ______.

11. The method of claim 10, wherein said vegetative propagation comprises regenerating a whole plant from said part of NUN 31503 WMW, wherein a representative sample of seed of said variety is deposited under Accession Number NCIMB ______.

12. The method of claim 10, wherein said part is a cutting, a cell culture or a tissue culture.

13. A vegetative propagated plant, or a part of said propagated plant, propagated from the plant of claim 1 or the plant part of claim 2 wherein the vegetative propagated plant has all of the physiological and morphological characteristics of the plant of NUN 31503 WMW, or one characteristic that is different from NUN 31503 WMW, when the characteristics are determined at the 5% significance level for plants grown under the same conditions, and wherein a representative sample of seed of NUN 31503 WMW is deposited under Accession Number NCIMB ______.

14. A Watermelon plant having one, two or three physiological and/or morphological characteristics which are different from those of the plant of claim 1 and which otherwise has all the physiological and morphological characteristics of the plant of claim 1, when determined at the 5% significance level for plants grown under the same environmental conditions.

15. The plant of claim 14, where the different characteristic is effected by a mutation or by transformation.

16. The plant of claim 1 further comprising a single locus conversion, wherein said plant has all or all but one, two or three of the morphological and physiological characteristics of the plant of NUN 31503 WMW, wherein a representative sample of seed of said variety is deposited under Accession Number NCIMB ______, when said characteristics are determined at the 5% significance level for plants grown under the same environmental conditions when grown under the same environmental conditions, optionally wherein the single locus conversion confers a trait selected from the group consisting of male sterility, herbicide tolerance, insect resistance, pest resistance, disease resistance, environmental stress tolerance, modified carbohydrate metabolism and modified protein metabolism.

17. A doubled haploid cell or plant made from haploid cells of NUN 31503 WMW wherein a representative sample of seed of NUN 31503 WMW is deposited under Accession Number NCIMB ______.

18. Use of the cell or plant of claim 17 in a method for generating parental lines of NUN 31503 WMW.

19. A method of producing a combination of parental lines of the plant of claim 1 comprising the steps of
a. defining a set of genetic markers that are present in a heterozygous form in NUN 31503 WMW
b. producing doubled haploid lines from NUN 31503 WMW:
c. genetically characterizing the doubled haploid lines to determine whether they are present in a first homozygous form or in a second homozygous form;
d. selecting at least one pair of doubled haploid lines that have complementary alleles for at least a subset of the genetic markers, wherein each member of the pair is suitable as a parental line for a hybrid organism.

20. A combination of parental lines generated by the method of claim 19.

21. A combination of two inbred plants which when crossed produce a seed or plant of claim 1.

22. A plant comprising the scion or rootstock of claim 2.

23. A container comprising a plant, plant part or seed of claim 1.

24. A food or feed product or a processed product comprising the plant part of claim 2.

25. Method for inducing mutation in the plant of claim 1 comprising the steps of
a. Exposing a seed, a plant or a plant part of NUN 31503 WMW to a mutagenic chemical or to radiation, wherein a representative sample of seed of NUN 31503 WMW is deposited under Accession Number NCIMB ______.
b. Selecting a seed, a plant or a plant part or a cell of NUN 31503 WMW having a mutation

26. A method of producing a Watermelon fruit, comprising:
a. Growing a plant of claim 1 until it sets fruit
b. Collecting the fruit of step a)
